(19) United States
(12) Patent Application Publication
(10) Pub. No.: US 2017/0347838 A1
(43) Pub. Date: Dec. 7, 2017

(54) FOOD PROCESSOR AND INSERT RECEPTACLE FOR ARRANGEMENT IN A FOOD PROCESSOR

(71) Applicant: Vorwerk & Co. lnterholding GmbH, Wuppertal (DE)

(72) Inventors: Stefan KRAUT-REINKOBER, Leverkusen (DE); Torsten LANG, Solingen (DE); Georg HACKERT, Bochum (DE); Andres SAUERWALD, Bottrop (DE); Jutta SCHOMACHER, Wuelfrath (DE); Stefan HILGERS, Essen (DE)

(73) Assignee: Vorwerk & Co. lnterholding GmbH, Wuppertal (DE)

(21) Appl. No.: 15/538,332

(22) PCT Filed: Dec. 15, 2015
(86) PCT No.: PCT/EP2015/079715

§ 371 Date: Jul. 19, 2017

(30) Foreign Application Priority Data
Dec. 22, 2014 (DE) 10 2014 119 315.6

(51) Int. Cl. A47J 43/07; A47J 27/04; A47J 27/00; A47J 36/10; G06F 19/00; A61B 5/053

(52) U.S. Cl. CPC A47J 43/0727; G06F 19/3431; A61B 5/0537; A47J 43/0716; A47J 27/004; A47J 36/10; A47J 27/04; A47J 2027/043

(57) ABSTRACT

A food processor, in particular a mixing device, comprising a housing for accommodating a receptacle and a cover that closes the receptacle, and a locking mechanism for interlocking the receptacle with the cover; the locking mechanism includes a locking element, especially a locking roller, which can be moved relative to the receptacle and/or the cover from an unlocking position into a locking position and vice versa. In order to further develop food processors of the type, the locking element, apart from or in addition to interlocking the receptacle with the cover, is designed to have a subsidiary effect on the receptacle and/or the cover or to subsidiarily interact with the receptacle and/or the cover.

The invention relates to a food processor comprising a housing for accommodating a receptacle and a cover that closes the receptacle. The invention further relates in particular to such food processors which have a locking mechanism for interlocking the receptacle with the cover, wherein the locking mechanism includes a locking element, especially a locking roller, which can be moved relative to the receptacle and/or the cover from an unlocking position into a locking position and vice versa.

Food processors of the above-mentioned type are known in the prior art. These serve in particular in the household field for the production or preparation of foods, wherein the receptacle associated with the food processor is designed to receive foodstuffs. In the base region, a stirring device can be associated with the receptacle, which stirring device processes foods contained in the receptacle. Furthermore, it is also known to provide locking mechanisms which interlock the receptacle with the cover. In particular in the case of heatable receptacles, such as cooking receptacles, high pressures are in part reached within the receptacle during the cooking process. In connection with the stirring device, it can occur during operation of the food processor that a column of liquid acts on the underside against the cover which is placed onto the receptacle. Accordingly, the cover must be locked on the receptacle.

Proceeding from the above-mentioned prior art, it is an object of the invention to further develop food processors of this type and in particular to indicate further developments which expand the known functions of the food processor and therefore offer the user an additional benefit compared to the known food processors. In addition, it is aimed here to reduce the requirement for accessory devices necessary in addition to the food processor and therefore to offer a universal apparatus at a favourable cost.

To solve the above-mentioned problem, the invention firstly proposes a food processor, in particular a mixing device, comprising a housing for accommodating a receptacle and a cover that closes the receptacle, which has a locking mechanism for interlocking the receptacle with the cover, wherein the locking mechanism includes a locking element which can be moved relative to the receptacle and/or the cover from an unlocking position into a locking position and vice versa, and wherein the locking element, apart from or in addition to interlocking the receptacle with the cover is designed to have a subsidiary effect on the receptacle and/or the cover or to subsidiarily interact with the receptacle and/or the cover.

According to the invention, the subsidiary effect/interaction can be independent of the locking function of the food processor and therefore also independent of a current position of the locking element. Alternatively, however, it is also possible that the subsidiary effect on, or the interaction with the receptacle and/or the cover is coupled to the function of locking, in particular the unlocking position and/or locking position.

It is proposed that the locking element can be moved into a subsidiary position, which is arranged in relation to a locking position of the locking element from the unlocking position into the locking position so that the locking element firstly assumes the unlocking position, thereafter the locking position and simultaneously or subsequently to the locking position, assumes the subsidiary position. In the case where the subsidiary position is a separate third position in addition to the unlocking position and the locking position, the locking element can be designed and arranged so that the locking element firstly assumes the unlocking position, then the locking position and subsequently the subsidiary position. Consequently, a user of the food processor, if he does not require the subsidiary position, can carry out the usual handling of the locking element with respect to the unlocking position and the locking position. Alternatively, provision can be made that the locking element can be moved simultaneously into the locking position and the subsidiary position. For example, the locking element can be constructed having several parts, wherein a first partial region with respect to the locking position brings about a locking of receptacle and cover, whilst a second partial region of the locking element assumes the subsidiary position and in so doing observes a subsidiary function. Likewise, it is also possible that the locking element has an additional functional element, which can be moved separately into the subsidiary position. Here, the locking element, including the functional element, can be moved firstly into the locking position, whereupon the functional element can then be moved separately into the subsidiary position.

In the subsidiary position, the locking element can fulfil different functions, which bring about a subsidiary effect on or subsidiary interaction with the receptacle and/or the cover of the food processor. Different embodiments are proposed below.

According to a first embodiment of the invention, it is proposed that the locking element is in operative connection with a holding device, which in the subsidiary position is moved at least with a partial region in the direction of the interior of the receptacle or into the receptacle. The subsidiary position can therefore be used for example to introduce ingredients into the receptacle of the food processor by means of the holding device, which can be moved in the direction of the interior of the receptacle or into the receptacle. These ingredients can be applied either directly onto a partial region of the holding device and then moved into the receptacle, or can be held into the receptacle via an accessory device connected to the holding device, such as for example a tea- or herb bag. As the holding device or the accessory device are in operative connection with the movable locking element, the additives can be moved both into the receptacle or respectively in the direction of the receptacle and also in the opposite direction. Hereby, a device advantageously results by which for example teabags can be both introduced into the receptacle and also can be removed therefrom again. The holding device can be connected to the locking element for example by a snap-on connection, so that the locking position of the locking element is, at the same time, the subsidiary position in the sense of the invention.

Particularly advantageously, a time control is associated here with the locking mechanism, by means of which the locking element can be moved in a time-controlled manner into the subsidiary position, in particular remains in the subsidiary position for a predefined period of time and can be moved out from the subsidiary position again after the period of time has elapsed. According to this configuration, for example a teabag or a herb bag can be fastened to the holding device, which is in operative connection with the locking element. The holding device and the teabag (or respectively herb bag) arranged thereon points in the direction of the interior of the receptacle, but in the unlocking position and locking position of the locking element does not come in contact with the foods or liquids contained in the receptacle. Only with a movement of the locking element into the subsidiary position is the teabag or respectively herb bag let down into the interior of the receptacle to such an extent that a contact occurs between the tea or respectively the herbs and the content of the receptacle. As soon as the locking element is moved into the subsidiary position, advantageously a stopwatch can be started, which indicates the period of time in which the locking element is already situated in the subsidiary position. As soon as a predetermined period of time has elapsed, the time measurement is stopped and the locking element is moved back again from the subsidiary position into the unlocking position (or the locking position) so that via the holding device, which is in operative connection with the locking element, at the same time also the tea or the herbs are removed again from the receptacle or respectively from the content situated therein.

Advantageously, a temperature control can be associated with the locking mechanism, by means of which the locking element can be moved into the subsidiary position in a temperature-controlled manner, in particular on reaching a predefined temperature within the receptacle. The above-mentioned temperature control can be provided alternatively or additionally to the above-mentioned time control. By means of the temperature control it is possible, for example, that the locking element is only moved into the subsidiary position when a predefined temperature is reached within the receptacle. In so far as for example a teabag is arranged on the above-mentioned holding device, the subsidiary position can be assumed when water situated in the receptacle has reached boiling point. Starting from this moment, a time measurement can take place, which measures a brewing temperature suitable for the tea, for example 8 minutes. After this predetermined period of time has elapsed, the locking element can be moved back out from the subsidiary position, so that the teabag is removed again from the water.

Alternatively to the above-mentioned automation of the movement of the locking element into the subsidiary position, the movement can of course also be carried out manually by the user of the food processor.

The invention proposes furthermore that the locking element is in operative connection with a closure device, which is designed to close and/or to open a cover opening, formed on the cover, as a function of the position of the locking element. According to this embodiment, with a movement of the locking element into the subsidiary position simultaneously a movement of the closure device—preferably arranged on the cover—is brought about. The extent of the movement can be dimensioned here so that the cover opening is only partially, or completely, closed. Thereby, it is possible to regulate steam flowing out of the receptacle through the cover opening. This is advantageous in particular when for example a cooking receptacle is arranged on the cover of the receptacle, in which foodstuffs are to be cooked by means of the steam rising up out from the receptacle. The vaporization rate and the amount of steam escaping from the receptacle can be influenced according to the opening position of the closure device.

It is proposed that the closure device has a shutter, in particular a sliding shutter, an iris shutter or a folding shutter. By means of this shutter, the cover opening can be enlarged or reduced. The configuration of the shutter as an iris shutter presents itself in particular, because the cover opening thereby is influenced symmetrically particularly advantageously with regard to its lateral edges. Alternatively, however, a sliding shutter or a folding shutter can also be used, which closes the cover opening from only one direction, or else two sliding shutters/folding shutters, which are able to be closed or respectively opened in opposite directions.

Also in connection with the closure device, it is possible that the subsidiary position can be assumed simultaneously or subsequently to the locking position. In particular, it remains possible that the user uses the locking mechanism as usual only in connection with the unlocking position and the locking position. Alternatively, however, it is also possible that, for example if he uses a cooking attachment on the cover, he additionally makes use of the subsidiary position, in order to utilize the function of the closure device for steam regulation.

Particularly advantageously, the closure device can also be used in connection with the previously explained time control and/or temperature control. For example, it is possible that the locking element is transferred from the locking position into the subsidiary position when a particular temperature is reached in the receptacle of the food processor. Then, the subsidiary position can remain in this subsidiary position for a particular period of time, by means of the time control, before the cover opening is freed again. The movement of the locking element from the unlocking position and/or locking position into the subsidiary position can be carried out either automatically or else manually by the user.

Furthermore, it is proposed that the locking element is designed to interlock the receptacle with an attachment, in particular a steaming attachment, arranged on the cover. Likewise, a locking can also be provided between the attachment and an attachment cover. In contrast to the locking position known in the prior art, a possibility is created here to lock the steaming attachment, which is only necessary in individual cases. The usual movement of the locking element from the unlocking position into the locking position for locking of receptacle and cover can be retained, wherein through a subsidiary position, if applicable independent thereof, a situation-dependent additional locking of the steaming attachment is possible. In the subsidiary position for example an attachment catch, arranged on the locking element, can engage into an attachment projection, corresponding in a form-fitting manner, of the steaming attachment, so that the steaming attachment is securely connected to the cover and/or to the receptacle. Likewise, provision can also be made that an attachment cover of the steaming attachment is additionally locked. Also in this respect, the time control and/or temperature control according to the invention is able to be used.

The invention furthermore proposes a food processor, in particular a mixing device, comprising a housing for accommodating a receptacle and a cover that closes the receptacle, wherein the food processor additionally has a measuring device for measuring physical parameters of a user of the food processor, and an evaluation device which has a data connection to the measuring device. According to the invention, the food processor is therefore extended by a measuring device for measuring physical parameters. The physical parameters can be, for example, a mass (colloquially: weight) or a body fat percentage of the user. The above-mentioned functions are usually determined by means of separate personal scales, wherein the user receives information concerning his mass or respectively his body fat percentage for measuring these physical parameters. By the invention, the disadvantage is overcome that the user receives measurement results from the separate personal scales, which he can not use directly. Usually, if the user wishes to lose weight, he must pay attention himself that for example he ingests a smaller amount of energy per day and, for this, chooses those types of foodstuffs or such an amount of foodstuff which lead to a reduction of the amount of energy received via the food and which therefore lead finally to a reduction of the body weight. By the invention, a solution is now created, in which the information concerning the physical parameters is passed on directly to the food processor. An evaluation device associated with the food processor is arranged to evaluate the information concerning the physical parameters and to use this in the preparation of foods. The evaluation device—like the measuring device also—can be a separate device connected to the food processor via a data connection. Advantageously, the evaluation device can also be integrated into the food processor.

Particularly advantageously, the measuring device is an external personal scales, which is connected to the evaluation device by means of a wireless or cable connection. Particularly advantageously, the measuring device is connected to the evaluation device via Bluetooth.

Advantageously, personal parameters of the user are stored in a data memory of the evaluation device, such as his/her gender, height and age, so that a normal weight or ideal weight can be determined. The current weight can be compared to this normal weight or ideal weight, wherein the difference can be calculated. In an analogous manner, a corresponding evaluation can likewise be carried out with regard to the body fat percentage of the user.

Advantageously, in addition a data memory is associated with the evaluation device, which has a recipe collection, wherein the evaluation device is arranged to select recipes from the recipe collection as a function of the measured physical parameters of the user, and to transmit these for presentation to a display of the food processor. The evaluation device therefore determines from the personal data and the measured physical parameters of the user the difference from a desired physical ideal or normal parameter and at the same time suggests recipes which are advantageous for achieving the target. For example, particular set menus can be compiled, which lead to a reduction of the body fat percentage or the body weight. In this respect, the food processor according to the invention fulfils three functions equally, namely the function of food preparation, the function of determining physical parameters and the function of practical assistance in achieving a desired physical target parameter.

Furthermore, the measuring device can have measuring electrodes arranged on the food processor, in particular on a locking mechanism for interlocking the receptacle with the cover, or on the housing, which measuring electrodes are arranged to measure an electrical resistance of the body of the user. The measuring device can therefore be integrated directly into the food processor. For example, the housing of the food processor or a locking mechanism of the food processor can have measuring electrodes, by means of which an electrical resistance of the body of the user can be measured. In particular, metallic coverings are suitable for this, which are arranged on the locking mechanism. Particularly advantageously, the user can use the measuring device by clasping these metallic coverings with the palms of his hands. In so doing, the electrical resistance of the body of the user is measured and for example the body fat percentage is indicated by means of the measurement result.

The evaluation device can be configured to determine a body fat percentage of the user by means of personal data of the user, stored in a data memory associated with the evaluation device, in particular gender, height and/or age, and the measured electrical resistance.

Furthermore, a data memory which contains a recipe collection can be associated with the evaluation device also in connection with the measuring electrodes which are integrated in the food processor, wherein the evaluation device is arranged to select recipes from the recipe collection as a function of the measured physical parameters of the user, and to determine these for presentation to a display of the food processor. Alternatively, the recipes can also be communicated to the user to a mobile terminal, for example a smartphone or a laptop, or to a PC.

The invention additionally relates to a food processor, in particular a mixing device, comprising a housing for accommodating a receptacle and a cover that closes the receptacle, which food processor has a sound converter for the detection of ambient noises, and an evaluation- and control device, connected to the sound converter, which is designed to vary the sound intensity of acoustic signals emitted by the food processor as a function of the type and/or sound intensity of the detected ambient noises. The sound converter can be, for example, a microphone, which is integrated into the housing, preferably in the front region facing the user of the apparatus. The sound converter, in particular the microphone, is designed to detect the ambient sound intensity—filtered if applicable—and to pass on information concerning this to an evaluation- and control device, in which the type and sound intensity of the ambient noises are evaluated and thereupon if applicable the sound intensity of signals belonging to the apparatus is increased or reduced. In so far as it is therefore particularly loud in the environment of the food processor, the acoustic signals of the food processor can be raised by means of the evaluation- and control device so that a user standing in front of the food processor can continue to understand the signals emitted by the food processor. Likewise, in an environment which is almost free of ambient noises, a reduction of the signal sound intensity of the food processor can be carried out, so that a user standing in front of the food processor is not startled when acoustic signals are emitted by the food processor. In addition to the sound intensity of the detected ambient noises, the evaluation- and control device can likewise also determine the type of ambient noises. Depending on whether the type of ambient noises is similar to the type of acoustic signals emitted by the food processor, or is of a totally different type—background music, for example—the sound intensity of the signals of the food processor can likewise be increased or reduced. With noises of a similar type, for example an increase of the emitted acoustic signals is recommended, whereas with ambient noises which are not of similar type, a less high sound intensity is sufficient for differentiation of the acoustic signals emitted by the food processor, in order to differentiate these acoustic signals from the ambient noises.

Likewise, the invention proposes a food processor, in particular a mixing device, comprising a housing for accommodating a receptacle and a cover that closes the receptacle, wherein the food processor has an evaluation- and control device and a hotplate connected therewith, wherein the evaluation- and control device is designed to vary the temperature of the hotplate as a function of a current preparation progress of a food which is to be prepared by the food processor relative to a corresponding recipe stored in a data memory. The hotplate here can be an external hotplate or else a hotplate or warming plate integrated into the food processor itself. Depending on the specifications of a recipe selected by the user, and the current preparation progress of the recipe, the hotplate can already be pre-heated to a specified temperature during the preparation, so that the hotplate already has the desired temperature at the moment at which this is actually required. The evaluation- and control device of the food processor can control the hotplate here both from a chronological point of view and also with regard to the desired temperature. In so far as the hotplate is an external hotplate, the latter can be connected to the food processor by means of a cable connection. In so far as the hotplate is an internal hotplate of the food processor, the food processor can have for example within the same housing a hotplate for the main receptacle, for example the stirring receptacle, and in addition a hotplate which serves for heating or keeping warm some ingredients or parts of the prepared food. Through this configuration according to the invention, the effective preparation time of a food is reduced, because the user of the food processor can already carry out different working steps in a parallel manner, for example the preheating of an ingredient and, at the same time, for example the mechanical treatment of a food contained in the main receptacle.

Finally, an insert receptacle, in particular a mixing receptacle, is proposed by the invention for arrangement in a food processor, wherein the insert receptacle is produced from card or composite card, or wherein the insert receptacle is produced from a plastic with a modulus of elasticity less than 5 kN/mm², in particular less than 0.5 kN/mm², in particular from polyethylene. According to the invention, the insert receptacle is produced from a material which is not suitable for the long-term use of the food processor, but rather for example permits only a one-time or few preparation steps of a food. The insert receptacle therefore serves as a disposable receptacle or disposable receptacle insert, which is disposed of after being used as intended. This type of insert receptacle can be provided only for particular preparation steps which are suitable in connection with the choice of material, for example a crushing of herbs. The advantage for the user consists in that the insert receptacle does not have to be cleaned, but rather can be disposed of completely. Here, the insert receptacle as such can be configured as a disposable object, or else the otherwise usual receptacle of the food processor is lined with a disposable insert.

For example a disposable insert is conceivable, which is inserted into a metal receptacle and therefore only has to have a small material thickness, because the stability is provided by the surrounding material of the receptacle. In particular in the case of disposable inserts, it is therefore proposed that this has as small a wall thickness as possible, for example less than 1 mm.

The invention is explained in further detail below with the aid of example embodiments. There are shown:

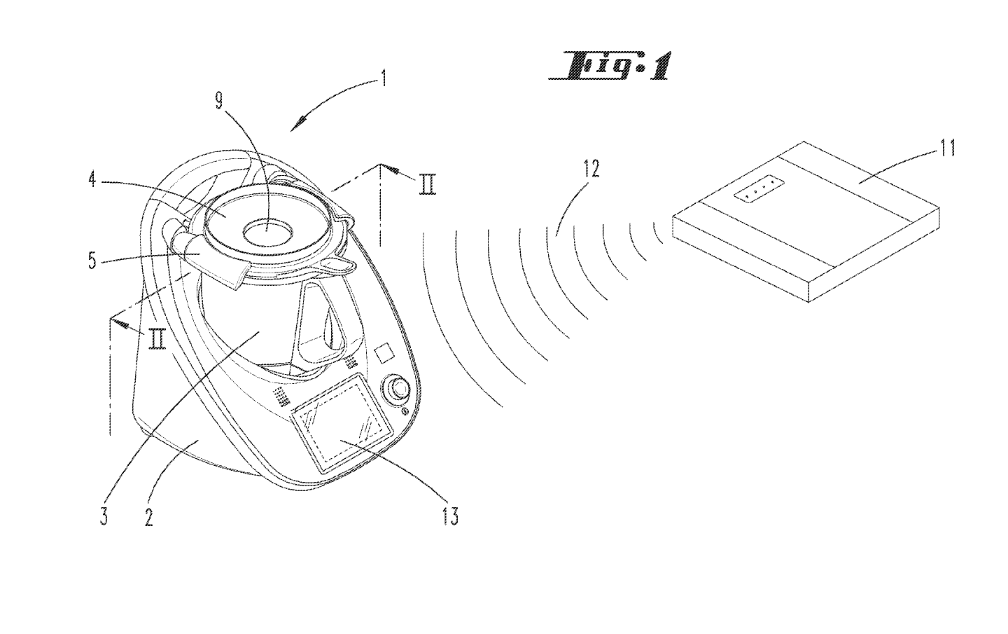

FIG. 1: a food processor with an external measuring device for measuring physical parameters,

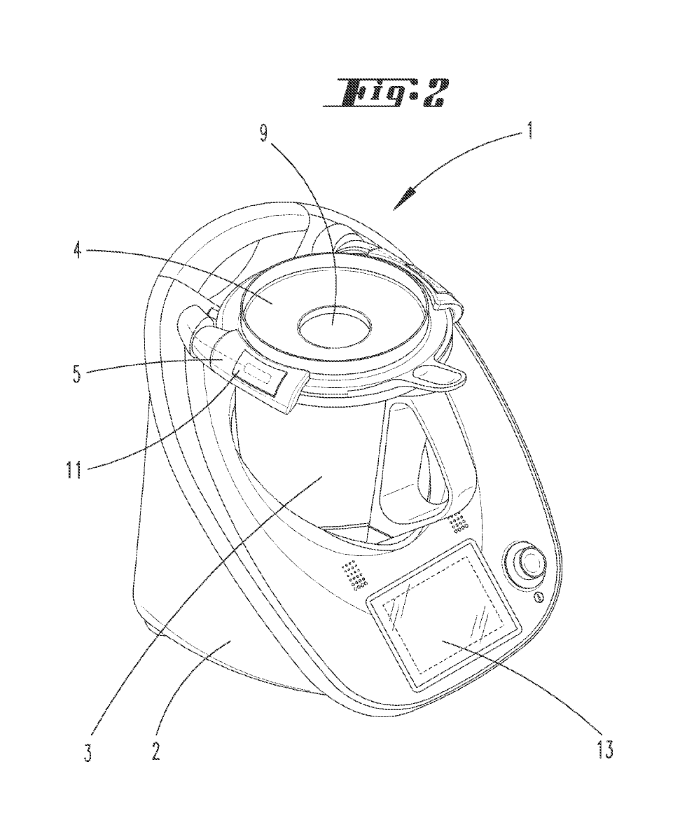

FIG. 2: a food processor with an internal measuring device for measuring physical parameters,

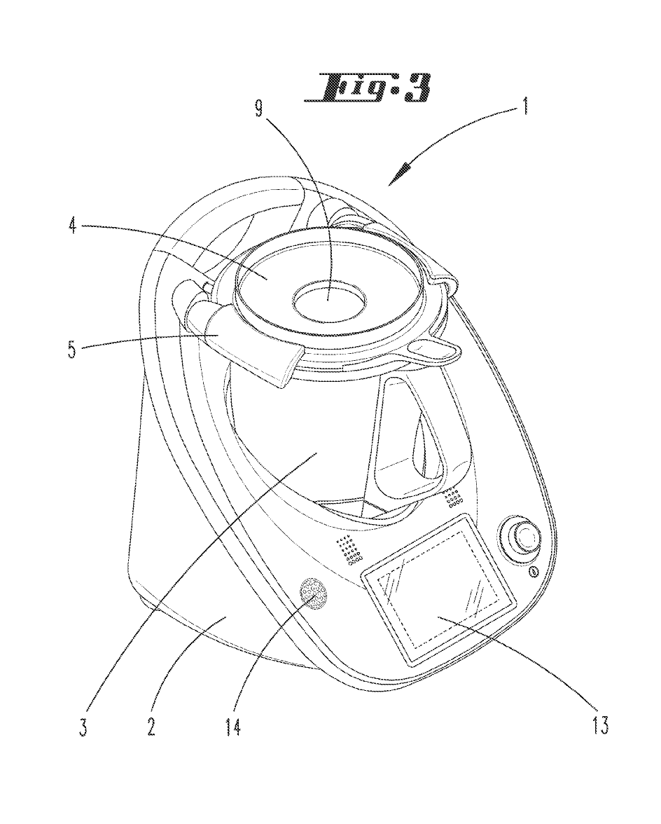

FIG. 3: a food processor with an integrated sound converter for the detection of ambient noises,

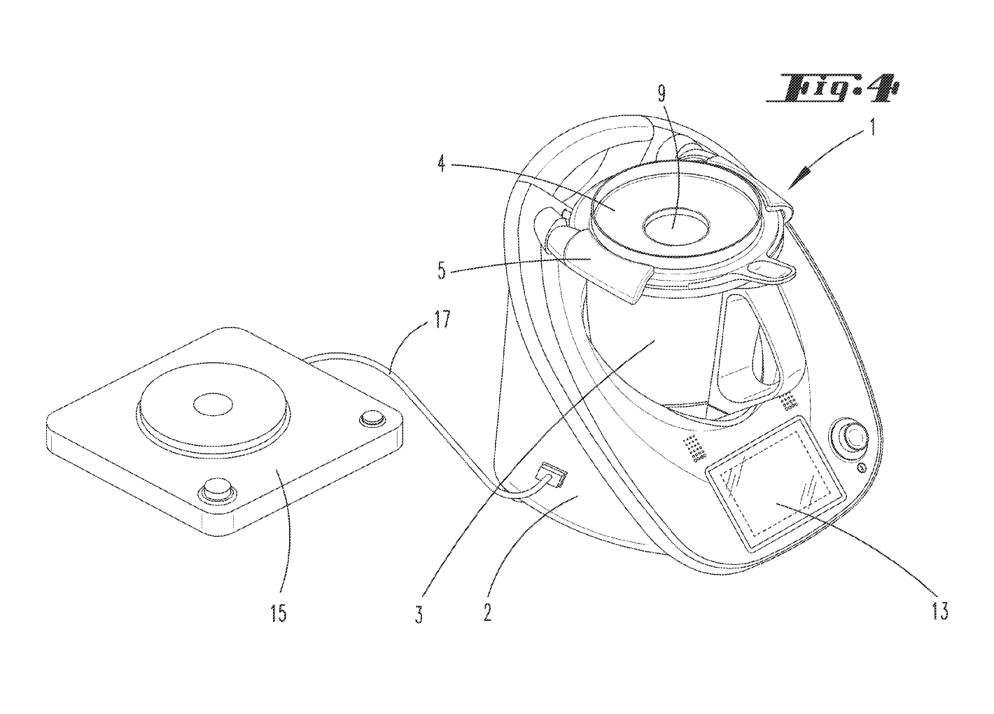

FIG. 4: a food processor with an external hotplate,

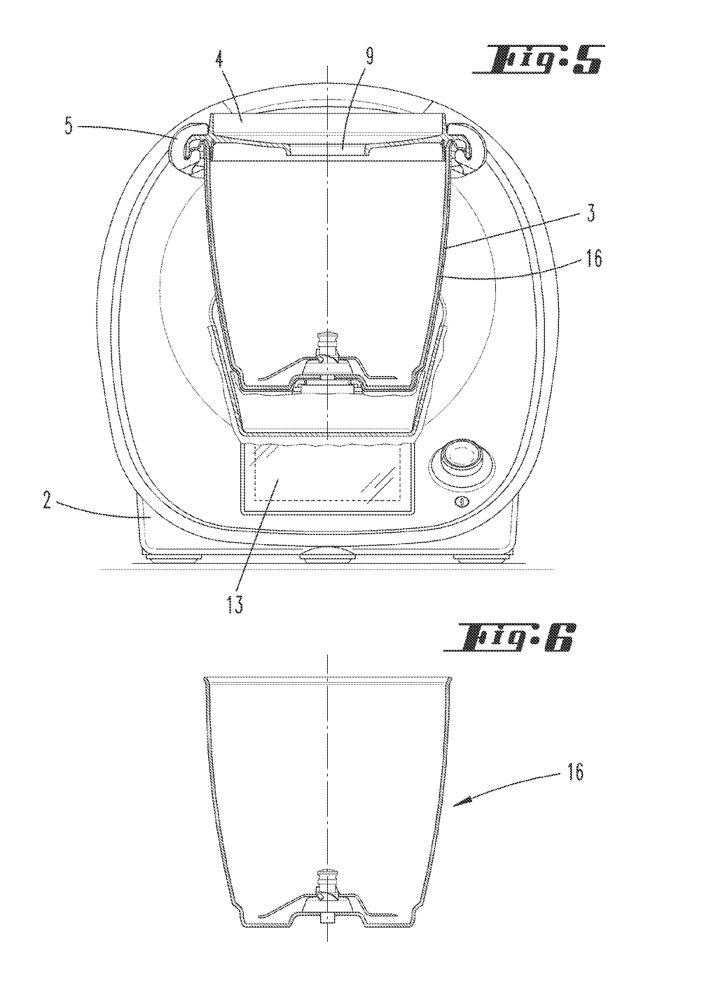

FIG. 5: a food processor with a receptacle in which an insert receptacle is arranged,

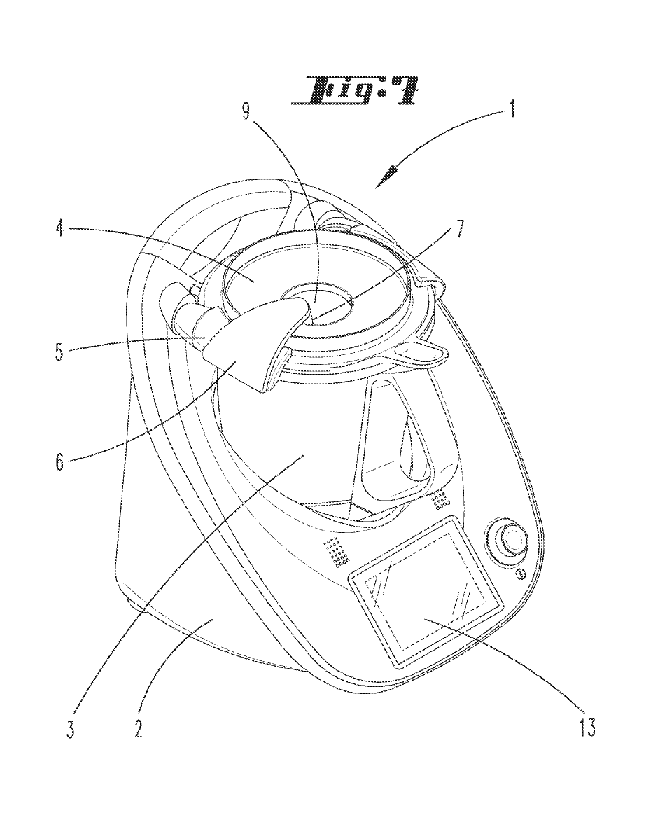

FIG. 6: an insert receptacle,

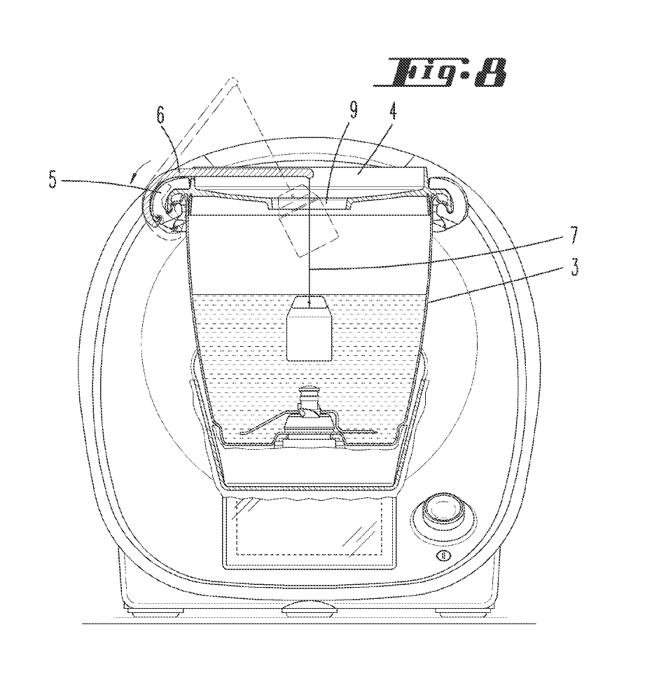

FIG. 7: a food processor with a locking mechanism according to a first embodiment, in perspective view,

FIG. 8: the food processor according to FIG. 7 in a cross-section,

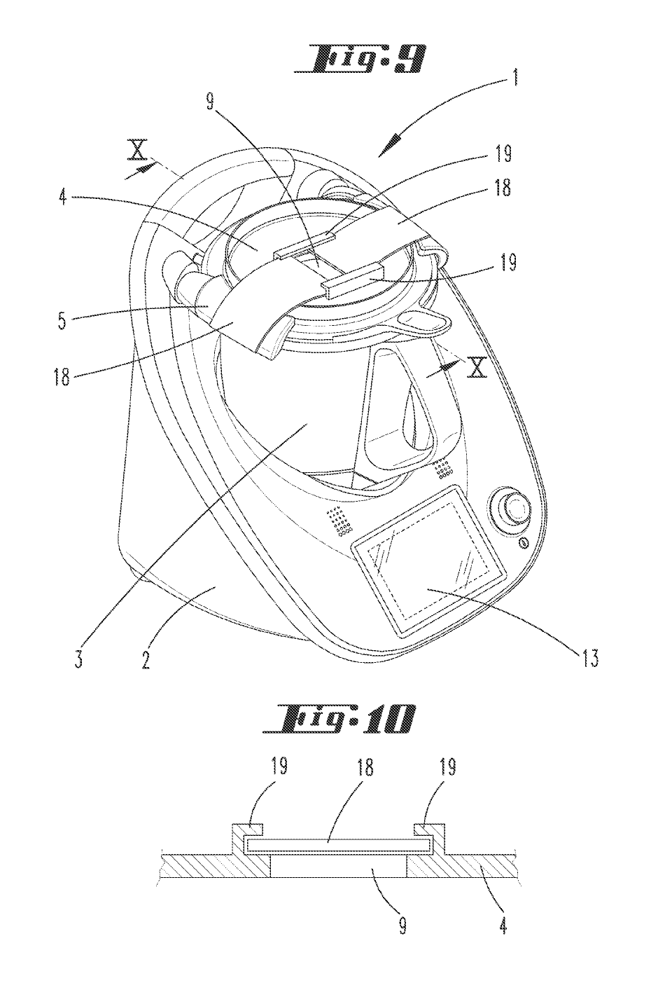

FIG. 9: a food processor with a locking mechanism according to a second embodiment in perspective view,

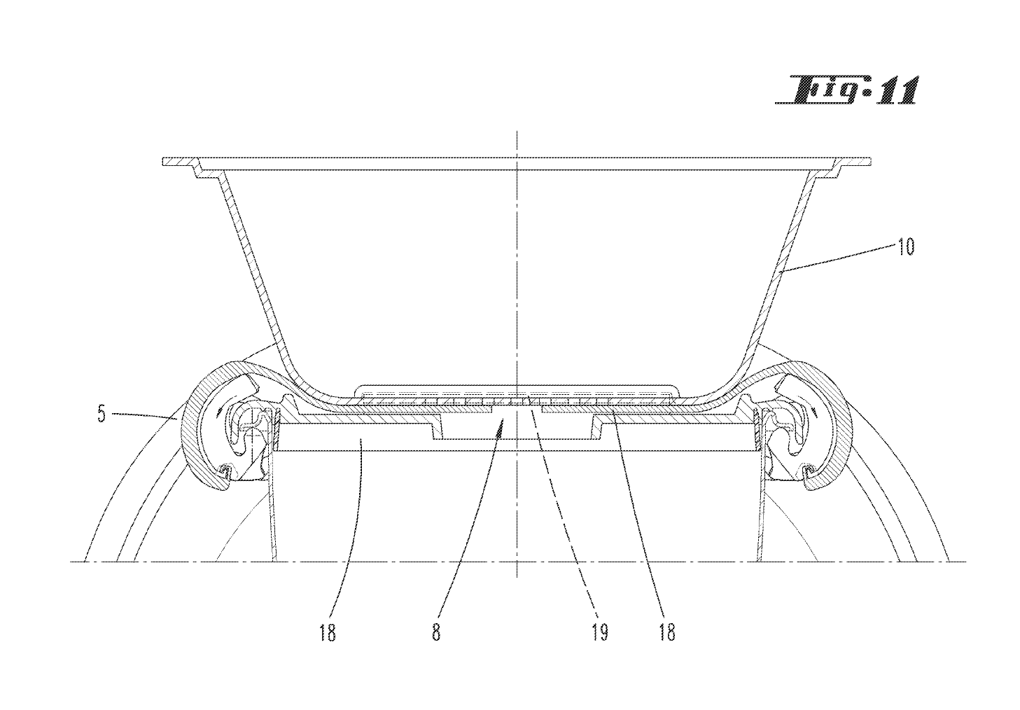

FIG. 10: the locking mechanism of the food processor illustrated in FIG. 9, in a cross-section,

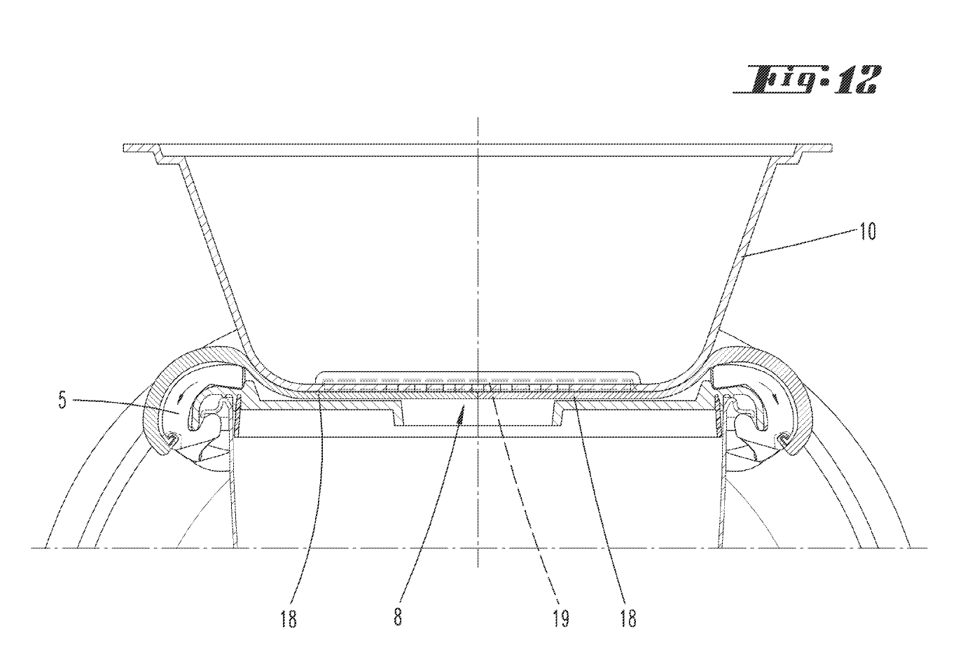

FIG. 11: the food processor according to FIG. 9 in a cross-section in a first position,

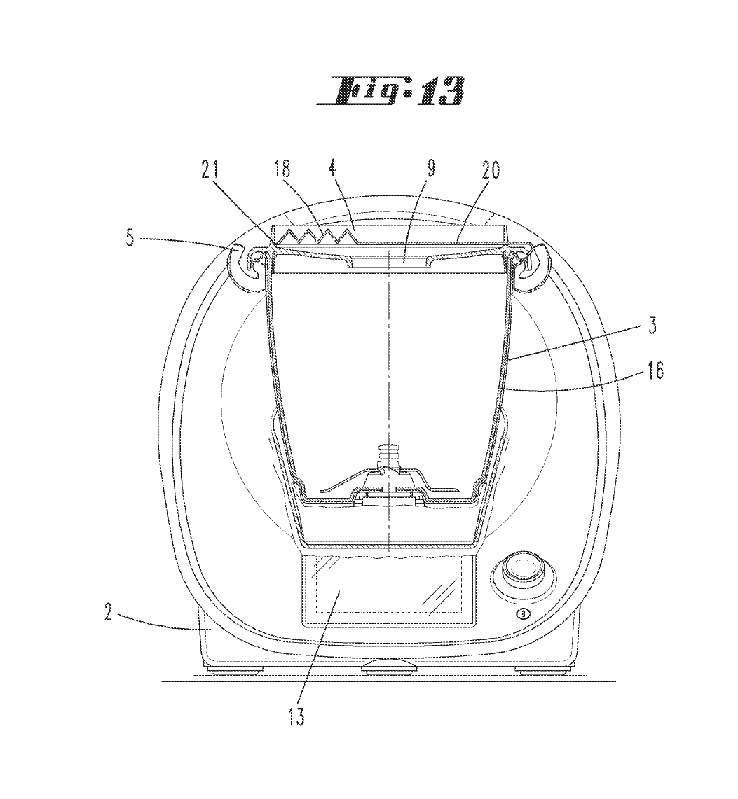

FIG. 12: the food processor according to FIG. 11 in a second position,

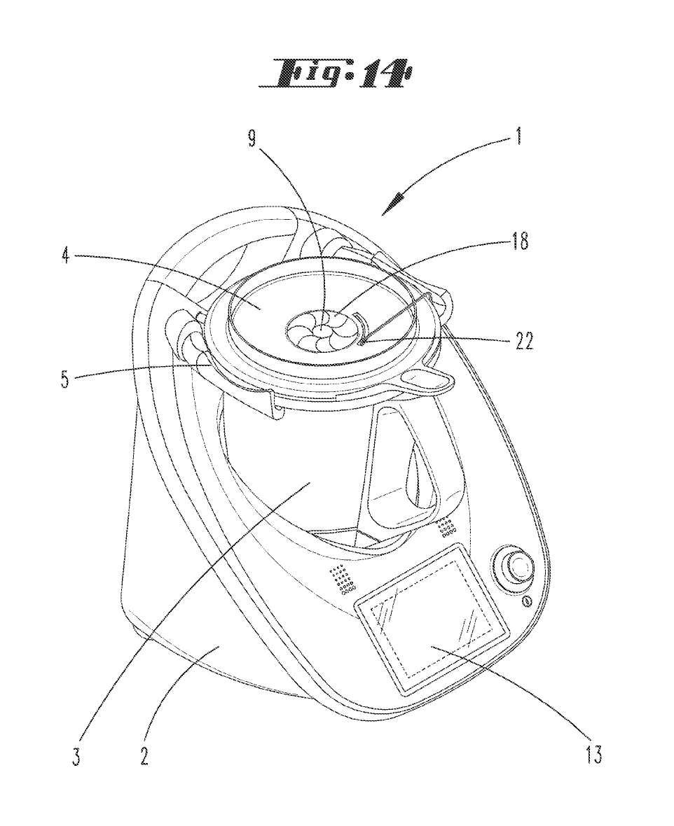

FIG. 13: a food processor with a locking mechanism according to a third embodiment,

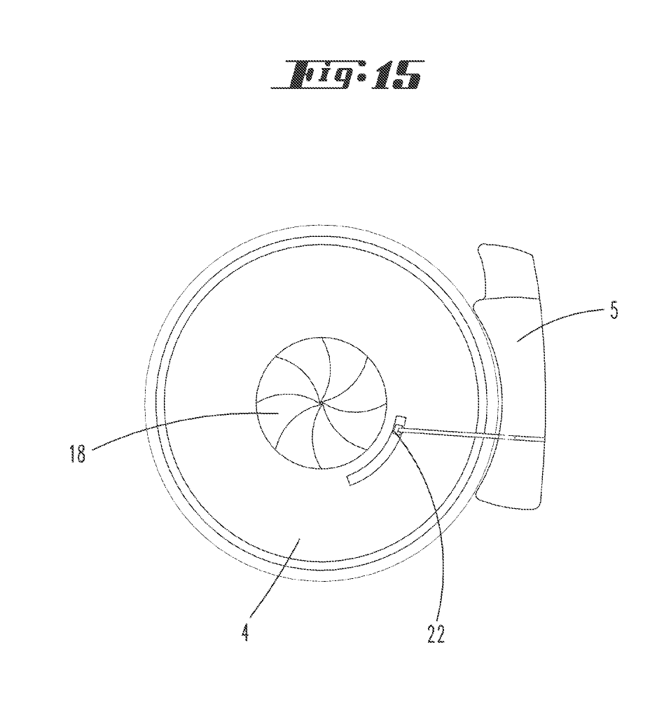

FIG. 14: a food processor with a locking mechanism according to a fourth embodiment, in perspective view,

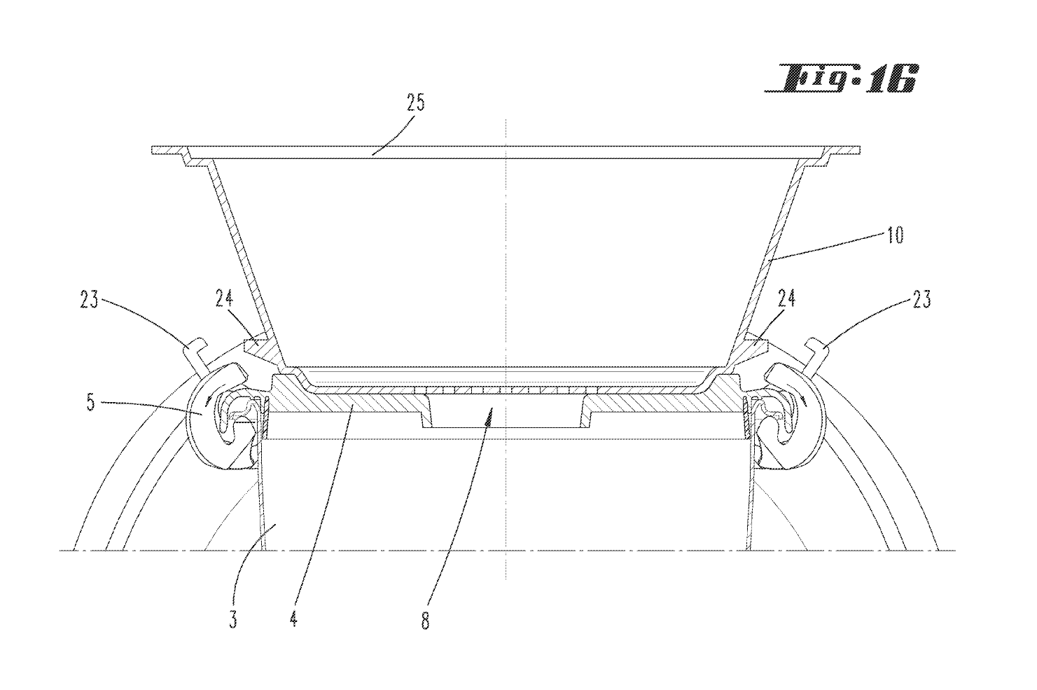

FIG. 15: the locking mechanism of the food processor according to FIG. 14, in a top view,

FIG. 16: a food processor with a locking mechanism according to a fifth embodiment.

The illustrated food processor 1 has a housing 2, on which a receptacle 3 and a cover 4 are arranged. The cover 4 closes the receptacle 3 and has in addition a cover opening 9, through which, if applicable, steam can escape into an attachment 10, in particular a steaming attachment, placed onto the cover 4. A locking mechanism is arranged on the housing 2, which locking mechanism has locking elements 5, here in the form of locking rollers. The locking elements 5 interlock the receptacle 3 with the cover 4. The locking elements 5 can be moved into different positions, for example from an unlocking position into a locking position. In addition, a display 13 is arranged on the housing 2, on which information can be communicated to the user.

FIG. 1 shows the food processor 1 additionally with an external measuring device 11, here a personal scales, which is connected to the food processor 1 by means of a data connection 12, here a wireless connection. The measuring device 11 is designed for the measuring of physical parameters, wherein the physical parameters can be the mass of the user, his body fat percentage or other parameters.

The invention according to FIG. 1 functions so that a user places himself onto the measuring device 11 for the measuring of physical parameters. The measuring device 11 determines the mass and the body fat percentage of the user and transmits this via the data connection 12 to an evaluation device of the food processor 1. The evaluation device compares the measured physical parameters to standard values or ideal values, which are deposited in a table. The standard or respectively ideal values take into account for example the gender, the height and the age of the user. The measured physical parameters are compared to this standard or ideal value and resulting therefrom a difference is calculated. In so far as the physical parameters lie for example above the standard or ideal value and the user's aim is to reduce these values, for example, the evaluation device of the food processor 1 selects recipes from a recipe collection, which are suitable for achieving the target parameters, and indicates these to the user for selection on the display 13. The indicated recipes can be, for example, reduced-fat or generally energy-reduced. The user can then select one of the recipes and prepare corresponding foods.

FIG. 2 shows alternatively a food processor 1 with a measuring device integrated into the locking elements 5. The measuring device 11 has metallic measuring electrodes, which are arranged to measure an electrical resistance of the body of the user. For this, the user grasps the locking elements 5 with his hands and triggers a corresponding measurement of the physical parameter, here for example of the body fat percentage. The measuring device 11 then transmits the measurement values to the evaluation device of the food processor 1 by means of a data connection 12, here for example a cable connection. The further evaluation and transmission to the user can take place as previously illustrated with respect to FIG. 1.

FIG. 3 shows a food processor 1 with a sound converter 14, namely a microphone. The sound converter 14 is arranged to detect ambient noises of the food processor 1 and to transmit the detected signals to an evaluation- and control device. The evaluation- and control device compares the detected signals to standard values and controls the sound intensity of the acoustic signals emitted from the food processor 1 as a function of the type and/or sound intensity of the detected ambient noises, so that the user of the food processor 1 can differentiate the food processor's own signals from the ambient noises.

FIG. 4 shows a food processor 1 with an external hotplate 15. The hot plate 15 is connected to the food processor 1 via a cable 17. An evaluation- and control device of the food processor 1 is designed so that, as a function of a current preparation progress of a food which is to be prepared with the food processor 1, it varies the temperature of the hotplate 15 relative to a corresponding recipe stored in a data memory. For example, a stew can currently be prepared in the receptacle 3, which in a subsequent preparation step is to be supplemented with fresh herbs. Before the current preparation step is completed, the hotplate 15 is preheated, so that the receptacle 3 can then be placed onto the hotplate 15 to keep the foods contained therein warm. Thereupon—simultaneously keeping the receptacle 3 warm—a crushing of fresh herbs can take place within a further receptacle 3, which is inserted into the housing 2 of the food processor. As soon as the herbs are crushed, they are added into the receptacle 3 to the stew, and the latter is removed from the hotplate 15 for further processing and inserted again into the housing 2 of the food processor 1.

FIG. 5 shows a food processor 1 comprising a receptacle 3, into which an additional insert receptacle 16 is inserted. The insert receptacle 16 is, for example, a plastic receptacle with a smaller wall thickness compared to the receptacle 3. In particular, the insert receptacle 16 is produced from a flexible polyethylene, so that it has no inherent stability without connection to the receptacle 3, which would be suitable for the preparation of foods. Owing to the small inherent stability, the insert receptacle 16 is clamped in the contact region between receptacle 3 and cover 4, so that the insert receptacle 16 is fastened to the receptacle 3.

FIG. 6 shows an insert receptacle 16, which has an inherent stability. The insert receptacle 16 is produced for example from a card and can either be inserted into the receptacle 3 or can entirely replace the receptacle 3 of the food processor 1. Such an insert receptacle 16 serves for example for the crushing of herbs and is suitable to tolerate several crushing steps without being destroyed. The insert receptacle 16 can have, in addition, a stirring device, or can be configured so that a partial region of the stirring device can be arranged thereon.

FIGS. 7 and 8 show a food processor 1 with two locking elements 5, wherein a holding device 6 is associated with one locking element 5. A partial region 7 of the holding device 6 is moved into the receptacle 3 and holds a teabag. The partial region 7 of the holding device 6 can be, for example, a thread- or wire element. The holding device 6 is clamped onto the locking element 5 and is in operative connection with the locking element 5 such that a movement of the locking element 5 in the direction of the interior of the receptacle 3 likewise leads to a movement of the holding device 6 in the same direction. The locking element can be moved as usual into an unlocking position, in which the receptacle 3 and the cover 4 are not interlocked with one another (illustrated in dashed lines), and into a locking position (here: at the same time, the subsidiary position), in which the receptacle 3 is interlocked with the cover 4. A time control can be associated here with the locking mechanism, by means of which the locking element 5 can be moved in a time-controlled manner into the locking position/subsidiary position, and remains there over a predefined period of time, and after the period of time has elapsed is moved into the unlocking position again. In so far as a teabag is arranged on the partial region 7 of the holding device 6—as illustrated—, said teabag can be moved in a time-controlled manner within a necessary period of time, for example a brewing time of 8 minutes, inside the receptacle 3, so that the teabag dips into a liquid which is present in the receptacle 3. After the period of time has elapsed, the teabag is removed from the liquid again. In addition, a temperature control can be carried out, which moves the locking element 5 in a temperature-controlled manner, in particular on reaching a predefined temperature within the receptacle 3, which for example indicates a reaching of the boiling temperature of the liquid contained in the receptacle 3. The time control and temperature control can therefore likewise be advantageously combined.

FIGS. 9 to 12 show a food processor 1, the cover 4 of which is constructed with a closure device 8. As a function of a position of the locking elements 5, a cover opening 9 arranged on the cover 4 can be closed or opened. The closure device 8 has two shutters 18 (sliding shutters) which are arranged on opposite partial regions with respect to the circumferential direction of the cover opening 9.

The shutters 18 are guided by shutter guides 19. Through the fact that the closure device 8 is in operative connection with the locking elements 5, the closure device 8 is also moved on a movement of the locking elements 5. In so far as the locking elements 5 are moved for example from an unlocking position into a locking position/subsidiary position, the closure device 8 is moved from an open position of the cover opening 9 into a closed position of the cover opening 9. Therefore, a closure of the cover opening 9 can be brought about advantageously in connection with a time control and/or temperature control, in order in a targeted manner to let, or not let, steam, which escapes out from the receptacle 3, into an attachment 10 standing on the cover 4. For example, on reaching a particular temperature within the receptacle 3, a partial closure of the cover opening 9 can be carried out to control the steam rate, for which the locking elements 5 are moved into a first subsidiary position. In so far as no further inlet of steam into the attachment 10 is desired, the locking elements 5 can be moved into a second subsidiary position, in which the cover opening 9 is completely closed.

As shown in the figures, the closure device 8, in particular a partial region of the shutters 18, can be clamped onto the locking elements 5 or otherwise engaged with these, wherein an opposite partial region of the shutters 18 lies between the guides 19 arranged on opposite circumferential sides of the cover opening 9. The guides 19 can be constructed here in one piece with the cover 4 or can be connected therewith by means of connection elements. On a rotation of the locking elements 5, at the same time a displacement of the shutters 18 takes place over the cover opening 9 or respectively a displacement of the shutters 18 away from the cover opening 9. The cross-sectional view of the closure device 8 shown in FIG. 10 shows the hook shape of the guides 19, which provides for a maintaining of the desired displacement direction of the shutters 18 with respect to the cover opening 9.

FIG. 13 shows a further embodiment of a closure device 8, wherein the cover opening 9 is able to be closed by means of a shutter 18, which is constructed as a folding shutter. The shutter 18 lies in a marginal region of the cover 4 with respect to an open position of the cover opening 9 of the cover 4. The shutter 18 can be connected to the cover 4 by means of a fastening element 21 in this region. Preferably, on a side of the shutter 18 lying opposite the fastening element 21, the shutter 18 has a traction element 20, by means of which the shutter 18 can be drawn apart, i.e. unfolded. The traction element 20 can be a thread or a wire. The shutter 8 is produced from an elastic material, which on releasing of the traction element 20 is returned into the folded position, so that the cover opening 9 is opened again. In particular, a shape memory material is recommended as material for the shutter 18. In the embodiment which is shown, the traction element 20 is arranged on one of the locking elements 5, wherein the traction element 20 is guided beneath the locking element 5 so that the traction element 20 is released in the unlocking position of the locking element 5 and is drawn in the locking position/subsidiary position, so that the tractive force engages on the shutter 18, the latter folds apart is therefore guided over the cover opening 9. As soon as the locking element 5 is unlocked again, the tractive force onto the traction element 20 is dispensed with, so that the shutter 18 returns into its folded initial position.

FIGS. 14 and 15 show a further embodiment, in which the shutter 18 is configured as an iris shutter. The shutter 18 is introduced into the cover opening 9 of the cover 4 and is in operative connection with an actuating element 22, which in turn is in operative connection with a locking element 5 of the food processor 1. The actuating element 22 and the locking element 5 are advantageously connected to a traction element, which can be a thread, a wire or similar. On a transferring of the locking element 5 from an unlocking position into the locking position (or, if applicable, an additional subsidiary position), the traction element is also moved and acts on the actuating element 22, which is configured here as a slide control closing the shutter 18. Therefore, a closing of the cover opening 9 is brought about. The cover opening 9 can be transferred manually by the user into an open position again, wherein the user engages on the slide control of the actuating element 22.

FIG. 16 shows a cooking attachment locking, which brings about an interlocking of the receptacle 3 of the food processor 1 with the attachment 10, here a steaming attachment. The locking elements 5 of the food processor 1 have attachment catches 23, which can be applied in a manner corresponding with regard to shape on attachment projections 24 of the attachment 10. The attachment catches 23 can either be securely connected to the locking elements 5 of the food processor 1, so that on a rotation of the locking elements 5 automatically a rotation of the attachment catches 23 occurs at the same time. Here, the subsidiary position, in which the receptacle 3 is interlocked with the attachment 10 is, at the same time, the locking position of the locking elements 5. Alternatively, however, it is also possible to configure the attachment catches 23 so as to be independently movable from the locking elements 5, so that independently of the locking position of the locking elements 5, a subsidiary position is able to be assumed, in which the attachment catches 23 interact with the attachment projections 24. Likewise, it is also possible to carry out an interlocking of the attachment 10 with the attachment cover 25, wherein for this purpose likewise corresponding attachment cover catches can be arranged on the locking element 5 of the food processor 1.

### LIST OF REFERENCE NUMBERS

1 food processor
2 housing
3 receptacle
4 cover
5 locking element
6 holding device
7 partial region
8 closure device
9 cover opening
10 attachment
11 measuring device
12 data connection
13 display
14 sound converter
15 hotplate
16 insert receptacle
17 cable
18 shutter
19 shutter guide
20 traction element
21 fastening element
22 fastening element
23 attachment catch
24 attachment projection
25 attachment cover

1-20. (canceled)

21. A food processor (1), comprising a housing (2) for accommodating a receptacle (3) and a cover (4) that closes the receptacle (3) and a locking mechanism for interlocking the receptacle (3) with the cover (4), wherein the locking mechanism includes a locking element (5), which can be moved relative to the receptacle (3) and/or the cover (4) from an unlocking position into a locking position and vice versa, wherein the locking element (5), apart from or in addition to interlocking the receptacle (3) with the cover (4), is designed to have a subsidiary effect on the receptacle (3) and/or the cover (4) or to subsidiarily interact with the receptacle (3) and/or the cover (4), wherein the locking element (5) can be moved into a subsidiary position, which is arranged in relation to a movement direction of the locking element (5) from the unlocking position into the locking position so that the locking element (5) firstly assumes the unlocking position, thereafter the locking position and, simultaneously or subsequently to the locking position, the subsidiary position, and either the subsidiary position is a separate third position or the locking element is constructed in several parts, wherein a first partial region brings about an interlocking of receptacle and cover with respect to the locking position, whilst a second partial region of the locking element assumes the subsidiary position and in so doing observes a subsidiary function, or the locking element has an additional functional element, which can be moved separately into the subsidiary position.

22. The food processor (1) according to claim 21, wherein the locking element (5) is in operative connection with a holding device (6), which in the subsidiary position is moved at least with a partial region in the direction of the interior of the receptacle or into the receptacle (3).

23. The food processor (1) according to claim 21, wherein the locking element (5) is in operative connection with a closure device (8), which is designed, as a function of the position of the locking element (5), to close and/or to open a cover opening (9) formed on the cover (4).

24. The food processor (1) according to claim 23, wherein the closure device (8) has a shutter (18).

25. The food processor (1) according to claim 21, wherein the locking element (5) is designed to interlock the receptacle (3) with an attachment (10), arranged on the cover (4).

26. The food processor (1) according to claim 21, wherein a time control is associated with the locking mechanism, by means of which the locking element (5) can be moved into the subsidiary position in a time-controlled manner.

27. The food processor (1) according to claim 21, wherein a temperature control is associated with the locking mechanism, by means of which the locking element (5) can be moved into the subsidiary position in a temperature-controlled manner.

28. A food processor (1), comprising a housing (2) for accommodating a receptacle (3) and a cover (4) that closes the receptacle (3), wherein a measuring device (11) for measuring physical parameters of a user of the food processor (1), and an evaluation device, which has a data connection (12) to the measuring device (11) are provided, wherein the measuring device (11) is an external personal scales, which is connected to the evaluation device by means of a wireless- or cable connection (12).

29. The food processor (1) according to claim 28, wherein the measuring device (11) is designed to determine a mass or a body fat percentage of the user.

30. A food processor (1), comprising a housing (2) for accommodating a receptacle (3) and a cover (4) that closes the receptacle (3), wherein a measuring device (11) for measuring physical parameters of a user of the food processor (1) and an evaluation device, which has a data connection (12) to the measuring device (11) are provided, characterized in that the measuring device (11) has measuring electrodes arranged on the food processor (1), or on the housing (2), which measuring electrodes are arranged to measure an electrical resistance of the body of the user.

31. The food processor (1) according to claim 30, wherein the evaluation device is designed to determine a body fat percentage of the user by means of personal data of the user, in particular gender, height and/or age, deposited in a data memory associated with the evaluation device, and the measured electrical resistance.

32. The food processor (1) according to claim 28, wherein a data memory is associated with the evaluation device, which has a recipe collection, wherein the evaluation device is arranged to select recipes from the recipe collection as a function of the measured physical parameters of the user, and to transmit them for presentation to a display (13) of the food processor (1).

33. A food processor (1), comprising a housing (2) for accommodating a receptacle (3) and a cover (4) that closes the receptacle (3), comprising a sound converter (14) for the detection of ambient noises of the food processor (1), and an evaluation- and control device connected to the sound converter (14), which is designed to vary the sound intensity of acoustic signals emitted by the food processor (1) as a function of the type and/or sound intensity of the detected ambient noises.

34. A food processor (1), comprising a housing (2) for accommodating a receptacle (3) and a cover (4) that closes the receptacle (3), comprising an evaluation- and control device and an external hotplate (15) connected therewith or a hotplate (15) provided additionally to a hotplate (15) for a main receptacle of the food processor within the same housing, wherein the evaluation- and control device is designed to vary the temperature of the external or additional hotplate (15) as a function of a current preparation progress of a food which is to be prepared with the food processor (1) relative to a corresponding recipe stored in a data memory.

35. A mixing receptacle, for arrangement in a food processor (1), wherein the mixing receptacle (16) is produced from card or composite card.

36. The mixing receptacle according to claim 35, wherein it has a wall thickness which is less than 1 mm.

37. An insert receptacle (16), for arrangement in a food processor (1), wherein the insert receptacle (16) is produced from a plastic having a modulus of elasticity less than 5 kN/mm2, in particular from polyethylene.

38. The insert receptacle (16) according to claim 37, wherein the receptacle (16) has a wall thickness which is less than 1 mm.

39. The insert receptacle (16) according to claim 37, wherein the insert receptacle (16) is designed for arrangement within a receptacle (3), in particular a mixing receptacle.
